(19) United States
(12) Patent Application Publication
(10) Pub. No.: US 2024/0025821 A1
(43) Pub. Date: Jan. 25, 2024

(54) PROCESS FOR CONVERTING OLEFINS TO DISTILLATE FUELS

(71) Applicant: UOP LLC, Des Plaines, IL (US)

(72) Inventors: Ashish Mathur, Gurugram (IN); Charles Luebke, Mt. Prospect, IL (US); Manuela Serban, Northbrook, IL (US); Den-Yang Jan, Elk Grove Village, IL (US); Eseoghene Jeroro, Chicago, IL (US); Hosoo Lim, Glenview, IL (US)

(21) Appl. No.: 17/872,714

(22) Filed: Jul. 25, 2022

(51) Int. Cl. C07C 2/08; C10G 59/00; C07C 5/03; C10G 69/12

(52) U.S. Cl. CPC C07C 2/08; C10G 59/00; C07C 5/03; C10G 69/126; C10G 2300/4081; C10G 2400/08; C10G 2400/04

(57) ABSTRACT

A process for dimerizing and oligomerizing olefins to distillate fuels which manages the dimerization exotherm by diluting it with paraffins which are inert in the dimerization. The olefin stream can also be split and fed to multiple dimerization reactors to further reduce the heat generated. The ethylene feed can also be cooled before entering the dimerization reactor. The paraffins can be obtained from saturating oligomerized effluent.

### FIELD

The field is the conversion of olefins to distillate. The field may particularly relate to dimerizing olefins and oligomerizing the dimerized olefins to distillate fuels.

### BACKGROUND

Ethylene can be dimerized into olefins such as C4, C6 and C8 olefins. Olefin oligomerization is a process that can oligomerize smaller olefins into larger olefins. More specifically, it can convert olefins including dimerized olefins into a distillates including jet fuel and diesel range products. The oligomerized distillate can be saturated for use as transportation fuels.

The dimerization reaction of ethylene is highly exothermic. The exotherm generated by ethylene dimerization can be difficult to manage.

Jet fuel is one of the few petroleum fuels that cannot be replaced easily by electrical motor systems because a high energy output is required to fuel planes which cannot be supplied with electric motors. Large incentives are currently available for green jet fuel in certain regions.

An efficient process is desired for converting ethylene to distillate fuels.

### BRIEF SUMMARY

We have formulated a process for dimerizing and oligomerizing olefins to distillate fuels which manages the ethylene dimerization exotherm by diluting it with paraffin stream which is inert in the dimerization. The olefin stream can also be split and fed to multiple dimerization reactors to further reduce the heat generated. The ethylene feed can also be cooled before entering the dimerization reactor. The paraffin stream can be obtained from saturating oligomerized effluent.

### BRIEF DESCRIPTION OF THE DRAWINGS

FIG. 1 is a schematic drawing of an oligomerization section of a process and apparatus of the present disclosure.

FIG. 2 is a schematic drawing of a hydrogenation section of a process and apparatus of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure. As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5”, “T90” or “T95” means the temperature at which 5 mass percent, 90 mass percent or 95 mass percent, as the case may be, respectively, of the sample boils using ASTM D-86 or TBP.

As used herein, the term “initial boiling point” (IBP) means the temperature at which the sample begins to boil using ASTM D-7169, ASTM D-86 or TBP, as the case may be.

As used herein, the term “end point” (EP) means the temperature at which the sample has all boiled off using ASTM D-7169, ASTM D-86 or TBP, as the case may be.

As used herein, the term “diesel” means hydrocarbons boiling in the range of an IBP between about 125° C. (257° F.) and about 175° C. (347° F.) or a T5 between about 150° C. (302° F.) and about 200° C. (392° F.) and the “diesel cut point” comprising a T95 between about 343° C. (650° F.) and about 399° C. (750° F.) using the TBP distillation method or a T90 between 280° C. (536° F.) and about 340° C. (644° F.) using ASTM D-86. The term “green diesel” means diesel comprising hydrocarbons not sourced from fossil fuels.

As used herein, the term “jet fuel” means hydrocarbons boiling in the range of a T10 between about 190° C. (374° F.) and about 215° C. (419° F.) and an end point of between about 290° C. (554° F.) and about 310° C. (590° F.). The term “green jet fuel” means jet fuel comprising hydrocarbons not sourced from fossil fuels.

### DETAILED DESCRIPTION

The process disclosed involves dimerizing an olefin stream comprising ethylene followed by further oligomerizing the ethylene dimers and ethylene oligomers. The resulting oligomers can be separated to provide a distillate stream which may be saturated to provide distillate fuels. A saturated stream may be recycled to ethylene dimerization as diluent to absorb the exotherm. Additionally, the olefin stream may be split and charged to several dimerization catalyst beds also to manage the exotherm. The dimerization product of a catalyst bed which may contain unconverted olefins, also passes to a downstream bed hence boosting overall per pass conversion. Additionally, dimerization products of upstream catalyst beds serve as additional diluent to absorb the exotherm in a downstream catalyst bed.

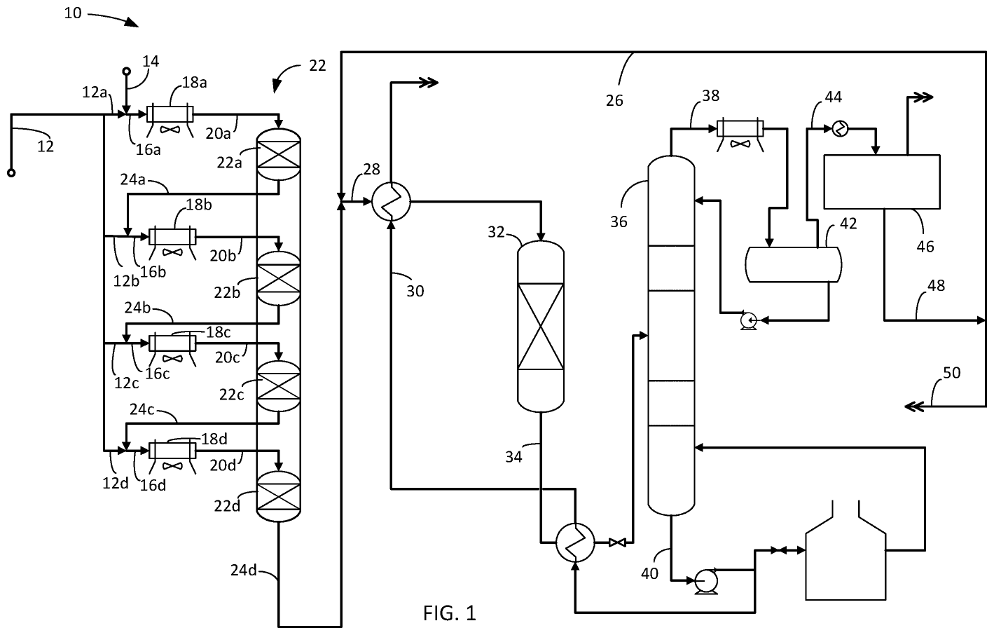

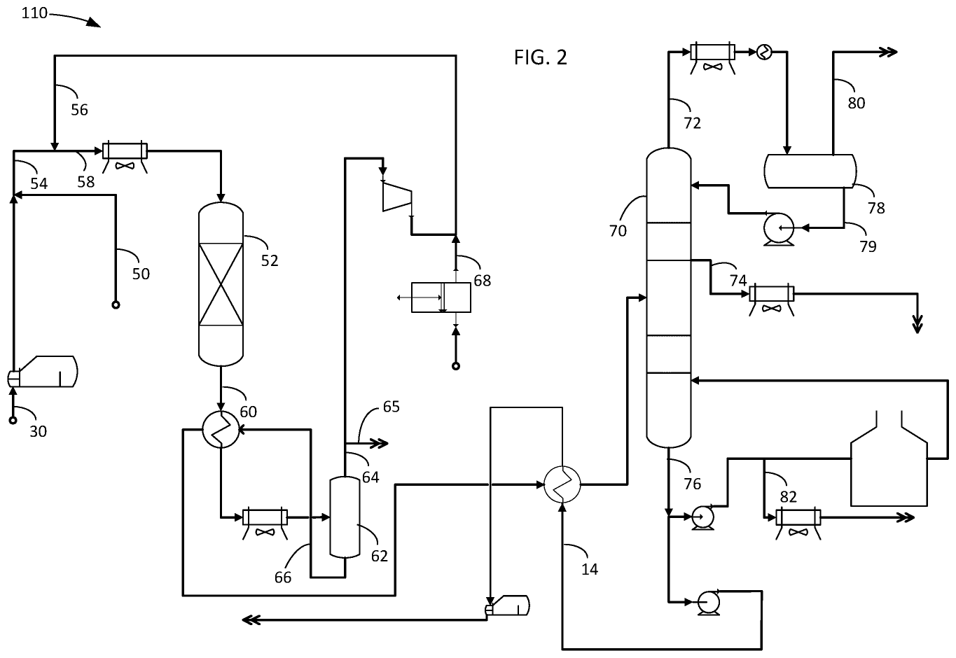

The process and apparatus may include an oligomerization section 10 in FIG. 1 and a hydrogenation section 110 in FIG. 2.

Turning to the oligomerization section 10 of FIG. 1, a charge olefin stream in line 12 is provided to the oligomerization section 10. The charge olefin stream may comprise substantial ethylene. The charge olefin stream may predominantly comprise ethylene. In an aspect, the charge olefin stream may comprise at least 95 mol % ethylene. The charge olefin stream in line 12 may be styled an ethylene stream. The olefin stream may be provided by the dehydration of ethanol or provided from a MTO unit. The charge olefin stream may be at a temperature of about 60° C. (140° F.) to about 150° C. (302° F.), preferably about 80° C. (176° F.) to about 100° C. (212° F.) and a pressure of about 5.6 MPag (800 psig) to about 8.4 MPag (1200 psig).

The olefin stream may be initially contacted with a dimerization catalyst to dimerize the ethylene to dimers and then contacted with an oligomerization catalyst to oligomerize dimerized ethylene. The dimerization reaction generates a large exotherm. For example, dimerization of ethylene can generate 612 kcal/kg (1100 BTU/lb) of heat. Consequently, this large exotherm must be managed.

Accordingly, the olefin stream in line 12 may be split into multiple olefin streams. In FIG. 1, the olefin stream is split into four separate streams: a first olefin stream in charge line 12a, a second olefin stream in charge line 12b, a third olefin stream in charge line 12c and a fourth or last olefin stream in charge line 12d. More or less separate multiple olefin streams may be used. Up to six olefin streams are readily contemplated. The charge olefin stream in line 12 may be split into equal aliquot multiple olefin streams. Alternatively, the charge olefin stream in line 12 may be split into unequal streams. For example, the charge olefin stream may be split into streams of ascending flow rates in which a subsequent olefin stream has a larger flow rate than a preceding stream. In an embodiment, the charge olefin stream is split into four streams of equal flow rates, each comprising 25 vol % of the charge olefin stream.

To manage the exotherm, the olefin stream may be diluted with a diluent stream to provide a diluted olefin stream to absorb the exotherm. The diluent stream may comprise a paraffin stream in a diluent line 14. The diluent stream in the diluent line 14 may be added to the charge olefin stream in line 12 before the charge olefin stream is split into multiple olefin streams. Preferably, the diluent stream is added to the first olefin stream in line 12a after the split into multiple olefin streams to provide a first diluted olefin stream in line 16a, so the diluent stream passes through all of dimerization reactions. Alternatively, the diluent stream may also be split into multiple streams with each diluent stream added to a corresponding olefin stream. The diluent stream may have a volumetric flow rate of about 2 to about 8 times and preferably about 3 to about 6 times the volumetric flow rate of the charge olefin stream. The first diluted olefin stream may comprise no more than 25 wt % olefins, suitably no more than 10 wt % olefins and preferably no more than 6 wt % olefins. The first diluted olefin stream may comprise no more than 25 wt % ethylene, suitably no more than 10 wt % ethylene and preferably no more than 6 wt % ethylene. The first diluted olefin stream in line 16a may be cooled in a first charge cooler 18a to provide a first cooled diluted olefin stream in line 20a and charged to a first bed 22a of dimerization catalyst in a dimerization reactor 22. The first cooled diluted olefin stream in line 20a may be charged at a temperature of about 54° C. (130° F.) to about 165° C. (329° F.) and a pressure of about 5.6 MPag (800 psig) to about 8.4 MPag (1200 psig).

The dimerization reactor 22 may comprise a series of dimerization catalyst beds 22a, 22b, 22c and 22d for charging with each multiple olefin stream 12a, 12b, 12c, and 12d, respectively. The dimerization reactor preferably contains four fixed dimerization catalyst beds 22a, 22b, 22c and 22d. It is also contemplated that each dimerization catalyst bed 22a, 22b, 22c and 22d may be in a dedicated dimerization reactor or multiple dimerization catalyst beds may be in two or more separate dimerization reactors. Up to six dimerization catalyst beds are readily contemplated. A parallel dimerization reactor may be used when the dimerization reactor 22 has deactivated during which the dimerization reactor 22 is regenerated in situ by combustion of coke from the catalyst.

The first cooled, diluted olefin stream may be charged to the first catalyst bed 22a in line 20a preferably in a down flow operation. However, upflow operation may be suitable. As dimerization of ethylene occurs in the first dimerization catalyst bed 22a, an exotherm is generated due to the exothermic nature of the ethylene dimerization reaction. Dimerization of the first olefin stream produces a first dimerized olefin stream in a first dimerized effluent line 24a at an elevated outlet temperature despite the cooling and dilution. The elevated outlet temperature is limited to between 50° C. (90° F.) and about 61° C. (110° F.) above the inlet temperature to the catalyst bed 22a.

The second olefin stream in line 12b may be diluted with the first dimerized olefin stream in line 24a removed from the dimerization reactor 22 to provide a second diluted olefin stream in line 16b. The first dimerized olefin stream in line 24a includes the diluent stream from diluent line 14 added to the first olefin stream in line 12a. The second diluted olefin stream may comprise no more than 25 wt % ethylene, suitably no more than 10 wt % ethylene and preferably no more than 6 wt % ethylene. The second diluted olefin stream in line 16b may be cooled in a second charge cooler 18b which may be located externally to the dimerization reactor 22 to provide a second cooled diluted olefin stream in line 20b and charged to a second bed 22b of dimerization catalyst in a dimerization reactor 22. The second cooled diluted olefin stream in line 20b may be charged at a temperature of about 54° C. (130° F.) to about 165° C. (329° F.) and a pressure of about 5.6 MPag (800 psig) to about 8.4 MPag (1200 psig). The second diluted olefin stream will include diluent and olefins from the first dimerized olefin stream. The olefins from the first dimerized olefin stream will dimerize in the second catalyst bed 22b. Dimerization of ethylene in the second olefin stream in the second bed 22b of dimerization catalyst produces a second dimerized olefin stream in a second dimerized effluent line 24b at an elevated outlet temperature. The elevated outlet temperature may be limited to between 50° C. (90° F.) and about 61° C. (110° F.) above the inlet temperature to the catalyst bed 22b.

The third olefin stream in line 12c may be diluted with the second dimerized olefin stream in line 24b removed from the dimerization reactor 22 to provide a third diluted olefin stream in line 16c. The second dimerized olefin stream in line 24b includes the diluent stream from diluent line 14 added to the first olefin stream in line 12a. The third diluted olefin stream may comprise no more than 25 wt % ethylene, suitably no more than 10 wt % ethylene and preferably no more than 6 wt % ethylene. The third diluted olefin stream in line 16c may be cooled in a third charge cooler 18c which may be located externally to the dimerization reactor 22 to provide a third cooled diluted olefin stream in line 20c and charged to a third bed 22c of dimerization catalyst in the dimerization reactor 22. The third cooled diluted olefin stream in line 20c may be charged at a temperature of about 54° C. (130° F.) to about 165° C. (329° F.) and a pressure of about 5.6 MPag (800 psig) to about 8.4 MPag (1200 psig). The third diluted olefin stream will include diluent and olefins from the second dimerized olefin stream. The olefins from the second dimerized olefin stream will dimerize in the third catalyst bed 22c. Dimerization of ethylene in the third olefin stream in the third bed 22c of dimerization catalyst produces a third dimerized olefin stream in a third dimerized effluent line 24c at an elevated outlet temperature. In an embodiment, the third dimerized olefin stream is a penultimate dimerized olefin stream and the third dimerized effluent line 24c is a penultimate dimerized effluent line 24c. The elevated outlet temperature is limited to between 50° C. (90° F.) and about 61° C. (110° F.) above the inlet temperature to the catalyst bed 22c.

The fourth olefin stream in line 12d may be diluted with the third or penultimate dimerized olefin stream in line 24c removed from the dimerization reactor 22 to provide a fourth diluted olefin stream in line 16d. The third or penultimate dimerized olefin stream in line 24c includes the diluent stream from diluent line 14 added to the first olefin stream in line 12a. The fourth diluted olefin stream may comprise no more than 25 wt % ethylene, suitably no more than 10 wt % ethylene and preferably no more than 6 wt % ethylene. The fourth diluted olefin stream in line 16d may be cooled in a fourth charge cooler 18d which may be located externally to the dimerization reactor 22 to provide a fourth cooled diluted olefin stream in line 20d and charged to a fourth bed 22d of dimerization catalyst in the dimerization reactor 22. The fourth cooled diluted olefin stream in line 20d may be charged at a temperature of about 54° C. (130° F.) to about 165° C. (329° F.) and a pressure of about 5.6 MPag (800 psig) to about 8.4 MPag (1200 psig). The fourth or last diluted olefin stream will include diluent and olefins from the third or penultimate dimerized olefin stream. The olefins from the third or penultimate dimerized olefin stream will dimerize in the fourth catalyst bed 22b. Dimerization of ethylene in the fourth olefin stream in the fourth bed 22d of dimerization catalyst produces a fourth dimerized olefin stream in a fourth dimerized effluent line 24d at an elevated outlet temperature. The elevated outlet temperature is limited to between 50° C. (90° F.) and about 61° C. (110° F.) above the inlet temperature to the catalyst bed 22d.

In an embodiment, the fourth olefin stream is a last olefin stream, the fourth dimerized olefin stream is a last dimerized olefin stream and the fourth dimerized effluent line 24d is a last dimerized effluent line 24d.

The dimerization reaction takes place predominantly in the liquid phase or in a mixed liquid and gas phase at a LHSV 0.5 to 10 hr⁻¹ on an olefin basis. We have found that a predominant fraction of ethylene in the olefin stream converts to higher olefins. Typically, at least 90 to about 95 mol % of ethylene will dimerize across a dimerization catalyst bed. The ethylene will initially dimerize over the catalyst to butenes.

The dimerization catalyst is preferably an amorphous silica-alumina base with a metal from either Group VIII and/or Group VIB in the periodic table using Chemical Abstracts Service notations. In an aspect, the catalyst has a Group VIII metal promoted with a Group VIB metal. Typically, the silica and alumina will only be in the base, so the silica-to-alumina ratio will be the same for the catalyst as for the base. The metals can either be impregnated onto or ion exchanged with the silica-alumina base. Co-mulling is also contemplated. Catalysts for the present invention may have a Low Temperature Acidity Ratio of at least about 0.15, suitably of about 0.2, and preferably greater than about 0.25, as determined by Ammonia Temperature Programmed Desorption (Ammonia TPD) as described hereinafter. Additionally, a suitable catalyst will have a surface area of between about 50 and about 400 m²/g as determined by nitrogen BET.

A preferred dimerization catalyst is described as follows. The preferred dimerization catalyst comprises an amorphous silica-alumina support. One of the components of the catalyst support utilized in the present invention is alumina. The alumina may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A particularly preferred alumina is available from Sasol North America Alumina Product Group under the trademark Catapal. This material is an extremely high purity alpha-alumina monohydrate (pseudo-boehmite) which after calcination at a high temperature has been shown to yield a high purity gamma-alumina. Another component of the catalyst support is an amorphous silica-alumina. A suitable silica-alumina with a silica-to-alumina ratio of 2.6 is available from CCIC, a subsidiary of JGC, Japan.

Another component utilized in the preparation of the catalyst utilized in the present invention is a surfactant. The surfactant is preferably admixed with the hereinabove described alumina and the silica-alumina powders. The resulting admixture of surfactant, alumina and silica-alumina is then formed, dried and calcined as hereinafter described. The calcination effectively removes by combustion the organic components of the surfactant but only after the surfactant has dutifully performed its function in accordance with the present invention. Any suitable surfactant may be utilized in accordance with the present invention. A preferred surfactant is a surfactant selected from a series of commercial surfactants sold under the trademark “Antarox” by Solvay S. A. The “Antarox” surfactants are generally characterized as modified linear aliphatic polyethers and are low-foaming biodegradable detergents and wetting agents.

A suitable silica-alumina mixture is prepared by mixing proportionate volumes silica-alumina and alumina to achieve the desired silica-to-alumina ratio. In an embodiment, about 75 to about 95 wt-% amorphous silica-alumina with a silica-to-alumina ratio of 2.6 and about 10 to about 20 wt-% alumina powder will provide a suitable support. In an embodiment, other ratios of amorphous silica-alumina to alumina may be suitable.

Any convenient method may be used to incorporate a surfactant with the silica-alumina and alumina mixture. The surfactant is preferably admixed during the admixture and formation of the alumina and silica-alumina. A preferred method is to admix an aqueous solution of the surfactant with the blend of alumina and silica-alumina before the final formation of the support. It is preferred that the surfactant be present in the paste or dough in an amount from about 0.01 to about 10 wt-% based on the weight of the alumina and silica-alumina.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried.

The paste or dough may be prepared in the form of shaped particulates, with the preferred method being to extrude the dough mixture of alumina, silica-alumina, surfactant and water through a die having openings therein of desired size and shape, after which the extruded matter is broken into extrudates of desired length and dried. A further step of calcination may be employed to give added strength to the extrudate. Generally, calcination is conducted in a stream of dry air at a temperature from about 260° C. (500° F.) to about 815° C. (1500° F.).

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as m; however, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

Typical characteristics of the amorphous silica-alumina supports utilized herein are a total pore volume, average pore diameter and surface area large enough to provide substantial space and area to deposit the active metal components. The total pore volume of the support, as measured by conventional mercury porosimeter methods, is usually about 0.2 to about 2.0 cc/gram, preferably about 0.25 to about 1.0 cc/gram and most preferably about 0.3 to about 0.9 cc/gram. Ordinarily, the amount of pore volume of the support in pores of diameter greater than 100 angstroms is less than about 0.1 cc/gram, preferably less than 0.08 cc/gram, and most preferably less than about 0.05 cc/gram. Surface area, as measured by the B.E.T. method, is typically above 50 m²/gram, e.g., above about 200 m²/gram, preferably at least 250 m²/gram, and most preferably about 300 m² gram to about 400 m²/gram.

To prepare the catalyst, the support material is compounded, as by a single impregnation or multiple impregnations of a calcined amorphous refractory oxide support particles, with one or more precursors of at least one metal component from Group VIII or VIB of the periodic table. The Group VIII metal, preferably nickel, should be present in a concentration of about 0.5 to about 15 wt-% and the Group VIB metal, preferably tungsten, should be present in a concentration of about 0 to about 12 wt-%. The impregnation may be accomplished by any method known in the art, as for example, by spray impregnation wherein a solution containing the metal precursors in dissolved form is sprayed onto the support particles. Another method is the multi-dip procedure wherein the support material is repeatedly contacted with the impregnating solution with or without intermittent drying. Yet other methods involve soaking the support in a large volume of the impregnation solution or circulating the support therein, and yet one more method is the pore volume or pore saturation technique wherein support particles are introduced into an impregnation solution of volume just sufficient to fill the pores of the support. On occasion, the pore saturation technique may be modified, so as to utilize an impregnation solution having a volume between 10 percent less and 10 percent more than that which will just fill the pores.

If the active metal precursors are incorporated by impregnation, a subsequent or second calcination at elevated temperatures, as for example, between 399° C. (750° F.) and 760° C. (1400° F.), converts the metals to their respective oxide forms. In some cases, calcinations may follow each impregnation of individual active metals. A subsequent calcination yields a catalyst containing the active metals in their respective oxide forms.

A preferred dimerization catalyst of the present invention has an amorphous silica-alumina base impregnated with 0.5-15 wt-% nickel in the form of 3.175 mm (0.125 inch) extrudates and a density of about 0.45 to about 0.65 g/ml. It is also contemplated that metals can be incorporated onto the support by other methods such as ion-exchange and co-mulling.

The dimerization catalyst can be regenerated upon deactivation. Suitable regeneration conditions include subjecting the catalyst, for example, in situ, to hot air at 500° C. for 3 hours. To facilitate regeneration without downtime, a swing bed arrangement may be employed with an alternative dimerization reactor. The regeneration gas may comprise air with an increased or decreased concentration of oxygen. Activity and selectivity of the regenerated catalyst is comparable to fresh catalyst.

The last dimerized olefin stream in the last dimerized effluent line 24d with an increased concentration of ethylene dimers and oligomers compared to the charge olefin stream in line 12 is mixed with an oligomer recycle stream in line 26 to provide a charge oligomerization stream in line 28. The charge oligomerization stream is heated by heat exchange with a net olefin splitter bottoms stream in line 30 and charged to an oligomerization reactor 32 at a temperature of about 204° C. (400° F.) to about 265° C. (509° F.) at a pressure of about 5.6 MPa (800 psig) to about 8.4 MPa (1200 psig).

The oligomerization reactor 32 may be in downstream communication with the dimerization reactor 22. The oligomerization reactor 32 preferably operates in a down flow operation. However, upflow operation may be suitable. The charge oligomerization stream is contacted with the oligomerization catalyst causing the C2-C8 olefins to dimerize and trimerize to provide distillate range olefins. A predominance of the butenes in the charge oligomerization stream is oligomerized. In an embodiment, at least 99 mol % of butenes in the charge oligomerization stream are oligomerized. An oligomerized olefin stream with an increased average carbon number greater than the charge oligomerization olefin stream in line 28 exits the oligomerization reactor 32 in line 34.

The oligomerization catalyst may include a zeolitic catalyst. The zeolite may comprise between about 5 and about 95 wt % of the catalyst, for example between about 5 and about 85 wt %. Suitable zeolites include zeolites having a structure from one of the following classes: MFI, MEL, ITH, IMF, TUN, FER, BEA, FAU, BPH, MEI, MSE, MWW, UZM-8, MOR, OFF, MTW, TON, MTT, AFO, ATO, and AEL. 3-letter codes indicating a zeotype are as defined by the Structure Commission of the International Zeolite Association and are maintained at http://www.iza-structure.org/databases. UZM-8 is as described in U.S. Pat. No. 6,756,030. In a preferred aspect, the oligomerization catalyst may comprise a zeolite with a framework having a ten-ring pore structure. Examples of suitable zeolites having a ten-ring pore structure include TON, MTT, MFI, MEL, AFO, AEL, EUO and FER. In a further preferred aspect, the oligomerization catalyst comprising a zeolite having a ten-ring pore structure may comprise a uni-dimensional pore structure. A uni-dimensional pore structure indicates zeolites containing non-intersecting pores that are substantially parallel to one of the axes of the crystal. The pores preferably extend through the zeolite crystal. Suitable examples of zeolites having a ten-ring uni-dimensional pore structure may include MTT. In a further aspect, the oligomerization catalyst comprises an MTT zeolite.

The oligomerization catalyst may be formed by combining the zeolite with a binder, and then forming the catalyst into pellets. The pellets may optionally be treated with a phosphorus reagent to create a zeolite having a phosphorous component between 0.5 and 15 wt % of the treated catalyst. The binder is used to confer hardness and strength on the catalyst. Binders include alumina, aluminum phosphate, silica, silica-alumina, zirconia, titania and combinations of these metal oxides, and other refractory oxides, and clays such as montmorillonite, kaolin, palygorskite, smectite and attapulgite. A preferred binder is an aluminum-based binder, such as alumina, aluminum phosphate, silica-alumina and clays.

One of the components of the catalyst binder utilized in the present invention is alumina. The alumina source may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A suitable alumina is available from UOP LLC under the trademark VERSAL. A preferred alumina is available from Sasol North America Alumina Product Group under the trademark Catapal. This material is an extremely high purity alpha-alumina monohydrate (pseudo-boehmite) which after calcination at a high temperature has been shown to yield a high purity gamma-alumina.

A suitable oligomerization catalyst is prepared by mixing proportionate volumes of zeolite and alumina to achieve the desired zeolite-to-alumina ratio. In an embodiment, the MTT content may about 5 to 85, for example about 20 to 82 wt % MTT zeolite, and the balance alumina powder will provide a suitably supported catalyst. A silica support is also contemplated.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried. Extrusion aids such as cellulose ether powders can also be added. A preferred extrusion aid is available from The Dow Chemical Company under the trademark Methocel.

The paste or dough may be prepared in the form of shaped particulates, with the preferred method being to extrude the dough through a die having openings therein of desired size and shape, after which the extruded matter is broken into extrudates of desired length and dried. A further step of calcination may be employed to give added strength to the extrudate. Generally, calcination is conducted in a stream of air at a temperature from about 260° C. (500° F.) to about 815° C. (1500° F.). The MTT catalyst is not selectivated to neutralize acid sites such as with an amine.

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as m; however, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

With regard to the oligomerization reactor 32, process conditions are selected to produce a higher percentage of jet range olefins which, when hydrogenated in a subsequent step as will be described below, result in a desirable jet-range hydrocarbon product. In one exemplary embodiment, an MTT-type zeolite catalyst disposed on a high purity pseudo boehmite alumina substrate in a ratio of about 90/10 to about 20/80 and preferably between about 20/80 and about 50/50 is provided in a catalyst bed or more in the oligomerization reactor 32. The charge oligomerization stream in line 28 is heated and charged to the oligomerization reactor 32. To achieve the most desirable olefin product, the oligomerization reactor 32 is operated at a temperature from about 204° C. (400° F.) to about 260° C. (500° F.). The oligomerization reactor 70 is run at a pressure from about 2.1 MPa (300 psig) to about 7.6 MPa (1100 psig), and more preferably from about 4.9 MPa (710 psig) to about 6.9 MPa (1000 psig).

Oligomerization reactions are also exothermic in nature. The last dimerized olefin stream in line 24d includes the diluent stream from diluent line 14 added to the first olefin stream in line 12a and carried through the dimerization catalyst beds 22a-22d. The diluent stream is then transported into the oligomerization reactor 32 in line 28 to absorb the exotherm in the oligomerization reactor.

When the oligomerization reaction is performed according to the above-noted process conditions, a C4 olefin conversion of greater than or equal to about 95% is achieved, or greater than or equal to 97%. The resulting oligomerized olefin stream in line 34 includes a plurality of olefin products that are distillate range hydrocarbons.

The oligomerization catalyst can be regenerated upon deactivation. Suitable regeneration conditions include subjecting the oligomerization catalyst, for example, in situ, to hot air at 500° C. for 3 hours. To facilitate regeneration without downtime, a swing bed arrangement may be employed with an alternative oligomerization reactor. A regeneration gas stream may be admitted to the oligomerization reactor 32 requiring regeneration. The regeneration gas may comprise air with an increased or decreased concentration of oxygen. Activity and selectivity of the regenerated catalyst is comparable to fresh catalyst.

An oligomerized olefin stream in line 34 with an increased C9+ olefin concentration compared to the charge oligomerization olefin stream in line 28 is heat exchanged with an olefin splitter bottoms stream in line 30 let down in pressure and fed to olefin splitter column 36.

In the olefin splitter column 36 oligomers that boil lower than the jet range hydrocarbons, typically C8− hydrocarbons with atmospheric boiling points less than about 150° C., are separated in an olefin splitter overhead stream in an overhead line 38 from a bottoms stream in a bottoms line 40 comprising distillate-range C9+ hydrocarbons, typically C9-C22 olefins. The olefin splitter column 36 may be operated at a bottoms temperature of about 400° C. (750° F.) to about 427° C. (800° F.) and an overhead pressure of about 172 kPa (25 psig) to about 517 kPa (75 psig). It is envisioned that the olefin splitter column 36 may be two columns. The olefin splitter overhead stream may be cooled and a resulting condensate portion refluxed from an olefin splitter receiver 42 back to the olefin splitter column 36. A net vapor stream in a receiver overhead line 44 from the olefin splitter receiver 42 may be compressed up to oligomerization pressure in an off-gas compressor 46 to provide a light oligomer stream in line 48 either in vapor phase or in liquid phase after cooling. Alternatively, the olefin splitter overhead stream in the overhead line 38 may be fully condensed by cooling perhaps in an external refrigeration loop to provide a liquid light oligomer stream in line 48. The light oligomer stream in line 48 may be split between a light olefin drag stream in line 50 and the oligomer recycle stream in line 26 recycled to the oligomerization reactor 32. The light olefin drag stream in line 50 may comprise about 3 to about 15 wt % of the light oligomer stream in line 48. The light oligomer stream in line 48 may comprise about 50 to about 80 wt % light olefins. The oligomer recycle stream in line 26 be mixed with the last dimerized olefin stream in the last dimerized effluent line 24d to provide the charge oligomerization stream in line 28 for charge to the oligomerization reactor 32. It is also envisioned that the oligomer recycle stream in line 26 be mixed with the first diluted olefin stream in line 16a or divided up between the first through fourth diluted olefin streams in lines 16a-16d to dimerize unreacted ethylene.

The heavy olefin stream in the splitter bottoms line 40 may be split between a reboil stream that is reboiled and fed back to the olefin splitter column 36 and a heavy olefin stream in a net splitter bottoms line 30. The heavy olefin stream in the net bottoms line 30 is cooled by heat exchange with the oligomerized olefin stream in line 34 and then with the charge oligomerization stream in line 28 before it is transported to the hydrogenation section 110 in FIG. 2.

Turning to the hydrogenation section 110 in FIG. 2, the heavy olefin stream in the net olefin splitter bottoms line 30 from FIG. 1 comprising distillate-range C9+ oligomerized olefins may be hydrogenated to saturate the olefinic bonds in a hydrogenation reactor 52 to provide fuels. This step is performed to ensure the product motor fuel meets or exceeds the thermal oxidation requirements specified in ASTM D7566-10a for hydroprocessed synthesized paraffinic kerosene (SPK). Additionally, saturating the oligomerized heavy olefins will provide the paraffin stream that may be used as the diluent stream in line 14. The heavy olefin stream in line 30 may be cooled to produce steam and be combined with the light olefin drag stream comprising C2 to C8 olefins in line 50 also from FIG. 1 to produce a combined olefin stream in line 54. The combined olefin stream in line 54 may also be combined with a hydrogen stream in line 56 to provide a combined hydrogenation charge stream in line 58 which is cooled and charged to the hydrogenation reactor 52 at 125° C. (257° F.) to about 204° C. (400° F.) and 3.5 MPa (500 psig) to about 6.9 MPa (1000 psig). An excess of hydrogen may be employed to ensure complete saturation such as about 1.5 to about 2.5 of stoichiometric hydrogen.

Hydrogenation is typically performed using a conventional hydrogenation or hydrotreating catalyst, and can include metallic catalysts containing, e.g., palladium, rhodium, nickel, ruthenium, platinum, rhenium, cobalt, molybdenum, or combinations thereof, and the supported versions thereof. Catalyst supports can be any solid, inert substance including, but not limited to, oxides such as silica, alumina, titania, calcium carbonate, barium sulfate, and carbons. The catalyst support can be in the form of powder, granules, pellets, or the like.

In an exemplary embodiment, hydrogenation is performed in the hydrogenation reactor 52 that includes a platinum-on-alumina catalyst, for example about 0.5 wt % to about 0.9 wt % platinum-on-alumina catalyst. The hydrogenation reactor 52 converts the olefins into a paraffin product having the same carbon number distribution as the olefins, thereby forming distillate-range paraffins suitable for use as jet and diesel fuel.

The saturated heavy stream discharged from the hydrogenation reactor 52 in line 60 may be cooled by heat exchange with a saturated heavy liquid stream in a separator bottoms line 66 and fed to a hydrogenation separator 62. In the hydrogenation separator 62, the saturated heavy stream is separated into a hydrogenated separator vapor stream in an overhead line 64 and the saturated heavy liquid stream in the hydrogenation separator bottoms line 66. A purge in line 65 may be taken from the hydrogenated separator vapor stream in line 64 and the remainder may be compressed and combined with make-up hydrogen in line 68 to provide the hydrogen stream in line 56. The saturated heavy liquid stream in the bottoms line 66 may be heated by heat exchange with the saturated heavy stream in line 60 and the diluent stream in line 14 and fed to a jet fractionation column 70.

The saturated heavy liquid stream in the bottoms line 66 may be fed to the jet fractionation column 70 without undergoing prior stripping in a stripper column. Alternatively, a stripper column may be utilized upstream of the jet fractionation column 70. In the jet fractionation column 70, the saturated heavy liquid stream may be separated into an off-gas stream in an overhead line 72, a green jet stream in a side line 74 from a side of the jet fractionation column 70 and a green diesel stream in a bottoms line 76. The jet fractionation column 70 may be operated at a bottoms temperature of about 427° C. (800° F.) to about 482° C. (900° F.) and an overhead pressure of about 35 kPa (5 psig) to about 350 kPa (50 psig).

The jet fractionation overhead stream in the overhead line 72 may be cooled and a resulting condensate portion refluxed from a jet fractionation receiver 78 back to the jet fractionation column 70 in line 79 while a net off gas stream comprising C8− hydrocarbons is taken in a receiver overhead line 80 from the jet fractionation receiver 78. Most of the hydrocarbons in the net off gas stream in the receiver overhead line 80 are lighter hydrocarbons and can be used to fuel the reboiler for the jet fractionation column 70 and/or the olefin splitter column 36.

The green jet stream taken in the side line 74 comprises kerosene range C9-C17 hydrocarbons and may be cooled and taken as product meeting applicable SPK standards. In an alternative embodiment, the green jet stream may be taken from the condensate stream in line 79 from the jet fractionation receiver 78 instead of refluxing all of the condensate to the column. This green jet stream taken from line 79 would have to be stripped to remove light ends. In such an embodiment, no side line 74 would be taken to recover the green jet fuel stream.

The green diesel bottoms stream in the bottoms line 76 may be split between a reboil stream that is reboiled and fed back to the jet fractionation column 70, a green diesel product stream in line 82 and a diluent stream in line 14. The diluent stream in line 14 may be cooled by heat exchange with the separator bottoms line 66 and by steam generation and recycled back to be mixed with the olefin stream in line 12 in the oligomerization section 10 in FIG. 1, preferably the first olefin stream in line 12a, to provide the first diluted olefin stream in line 16a to absorb the exotherm in the dimerization reactor 22. The green diesel in the diluent line 14 is paraffinic, so it will be inert to the dimerization, oligomerization and hydrogenation reactions to which it may be subject. Both the jet fuel stream in the side line 74 and the diesel stream in line 82 can be cooled and fed to their respective fuel pools. The diesel stream will meet ASTM D975 standards for diesel.

Starting with ethylene, the disclosed process can efficiently produce green jet fuel and green diesel fuel that meets applicable fuel requirements while managing exothermic heat generation. Carbon recovery in the process can exceed 95%.

### SPECIFIC EMBODIMENTS

While the following is described in conjunction with specific embodiments, it will be understood that this description is intended to illustrate and not limit the scope of the preceding description and the appended claims.

A first embodiment of the invention is a process for oligomerizing an olefin stream comprising diluting the olefin stream with a paraffin stream to provide a diluted olefin stream; dimerizing the diluted olefin stream with a dimerization catalyst to produce a dimerized olefin stream; oligomerizing the dimerized olefin stream with an oligomerization catalyst to provide an oligomerized olefin stream. An embodiment of the invention is one, any or all of prior embodiments in this paragraph up through the first embodiment in this paragraph further comprising splitting the olefin stream into multiple olefin streams and diluting a first olefin stream of the multiple olefin streams with the paraffin stream to provide a first diluted olefin stream and dimerizing the first diluted olefin stream to produce a first dimerized olefin stream. An embodiment of the invention is one, any or all of prior embodiments in this paragraph up through the first embodiment in this paragraph further comprising cooling the first diluted olefin stream before dimerizing the olefin stream. An embodiment of the invention is one, any or all of prior embodiments in this paragraph up through the first embodiment in this paragraph further comprising diluting a second olefin stream of the multiple olefin streams with the first dimerized olefin stream to provide a second diluted olefin stream and dimerizing the second diluted olefin stream to produce a second dimerized olefin stream. An embodiment of the invention is one, any or all of prior embodiments in this paragraph up through the first embodiment in this paragraph further comprising cooling the second diluted olefin stream before dimerizing the second diluted olefin stream. An embodiment of the invention is one, any or all of prior embodiments in this paragraph up through the first embodiment in this paragraph further comprising diluting a last olefin stream of the multiple olefin streams with a penultimate dimerized olefin stream to provide a last diluted olefin stream and dimerizing the last diluted olefin stream to produce the dimerized olefin stream. An embodiment of the invention is one, any or all of prior embodiments in this paragraph up through the first embodiment in this paragraph further comprising saturating the oligomerized olefin stream to provide the paraffin stream. An embodiment of the invention is one, any or all of prior embodiments in this paragraph up through the first embodiment in this paragraph further comprising taking a heavy olefin stream from the oligomerized olefin stream and saturating the heavy olefin stream to produce a saturated heavy stream and taking the paraffin stream from the saturated heavy stream. An embodiment of the invention is one, any or all of prior embodiments in this paragraph up through the first embodiment in this paragraph further comprising taking a diesel stream from the saturated heavy stream and taking the paraffin stream from the diesel stream. An embodiment of the invention is one, any or all of prior embodiments in this paragraph up through the first embodiment in this paragraph wherein the olefin stream is predominantly ethylene. An embodiment of the invention is one, any or all of prior embodiments in this paragraph up through the first embodiment in this paragraph wherein the diluted olefin stream has no more than 6 wt % ethylene. An embodiment of the invention is one, any or all of prior embodiments in this paragraph up through the first embodiment in this paragraph further comprising recycling oligomerized olefins taken from the oligomerized olefin stream to the oligomerization step.

A second embodiment of the invention is a process for oligomerizing an olefin stream comprising splitting the olefin stream into multiple olefin streams; dimerizing a first olefin stream of the multiple olefin streams to produce a first dimerized olefin stream; dimerizing a last olefin stream of the multiple olefin streams to produce a dimerized olefin stream; and oligomerizing the dimerized olefin stream with an oligomerization catalyst to provide an oligomerized olefin stream. An embodiment of the invention is one, any or all of prior embodiments in this paragraph up through the second embodiment in this paragraph further comprising diluting the first olefin stream with a paraffin stream to provide a first diluted olefin stream and dimerizing the first diluted olefin stream to produce the first dimerized olefin stream. An embodiment of the invention is one, any or all of prior embodiments in this paragraph up through the second embodiment in this paragraph further comprising cooling the first diluted olefin stream before dimerizing the olefin stream. An embodiment of the invention is one, any or all of prior embodiments in this paragraph up through the second embodiment in this paragraph further comprising diluting a second olefin stream of the multiple olefin streams with the first dimerized olefin stream to provide a second diluted olefin stream and dimerizing the second diluted olefin stream to produce a second dimerized olefin stream. An embodiment of the invention is one, any or all of prior embodiments in this paragraph up through the second embodiment in this paragraph further comprising cooling the second diluted olefin stream before dimerizing the second diluted olefin stream. An embodiment of the invention is one, any or all of prior embodiments in this paragraph up through the second embodiment in this paragraph further comprising taking a heavy olefin stream from the oligomerized olefin stream and saturating the heavy olefin stream to produce a saturated heavy stream and taking the paraffin stream from the saturated heavy stream.

A third embodiment of the invention is a process for oligomerizing an olefin stream comprising splitting the olefin stream into multiple olefin streams; dimerizing a first olefin stream of the multiple olefin streams to produce a first dimerized olefin stream; and dimerizing a last olefin stream of the multiple olefin streams to produce a dimerized olefin stream. An embodiment of the invention is one, any or all of prior embodiments in this paragraph up through the third embodiment in this paragraph further comprising oligomerizing the dimerized olefin stream with an oligomerization catalyst to provide an oligomerized olefin stream.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for oligomerizing an olefin stream to produce distillate fuels comprising:
diluting an ethylene stream with a paraffin stream to provide a diluted ethylene stream;
dimerizing said diluted ethylene stream with a dimerization catalyst to produce a dimerized olefin stream comprising ethylene dimers and oligomers;
oligomerizing said dimerized olefin stream with an oligomerization catalyst to provide an oligomerized olefin stream.

2. The process of claim 1 further comprising splitting said ethylene stream into multiple ethylene streams and diluting a first ethylene stream of said multiple ethylene streams with said paraffin stream to provide a first diluted ethylene stream and dimerizing said first diluted ethylene stream to produce a first dimerized olefin stream.

3. The process of claim 2 further comprising cooling said first diluted ethylene stream immediately before dimerizing said first diluted ethylene stream.

4. The process of claim 2 further comprising diluting a second ethylene stream of said multiple ethylene streams with said first dimerized olefin stream to provide a second diluted ethylene stream and dimerizing said second diluted ethylene stream to produce a second dimerized olefin stream.

5. The process of claim 4 further comprising cooling said second diluted ethylene stream before dimerizing said second diluted ethylene stream.

6. The process of claim 4 further comprising diluting a last ethylene stream of said multiple ethylene streams with a penultimate dimerized olefin stream to provide a last diluted ethylene stream and dimerizing said last diluted ethylene stream to produce said dimerized olefin stream.

7. The process of claim 1 further comprising saturating said oligomerized olefin stream to provide said paraffin stream.

8. The process of claim 7 further comprising taking a heavy olefin stream from said oligomerized olefin stream and saturating said heavy olefin stream to produce a saturated heavy stream and taking said paraffin stream from said saturated heavy stream.

9. The process of claim 8 further comprising taking a diesel stream from said saturated heavy stream and taking said paraffin stream from said diesel stream.

10. The process of claim 1 wherein said ethylene stream is predominantly ethylene.

11. The process of claim 1 wherein said diluted ethylene stream has no more than 6 wt % ethylene.

12. The process of claim 1 further comprising recycling oligomerized olefins taken from said oligomerized olefin stream to said oligomerization step.

13. A process for oligomerizing an olefin stream to produce distillate fuels comprising:
splitting an ethylene stream into multiple ethylene streams;
diluting a first ethylene stream of said multiple ethylene streams with a paraffin stream to provide a first diluted ethylene stream;
dimerizing a first ethylene stream of said multiple ethylene streams to produce a first dimerized olefin stream comprising ethylene dimers and oligomers;
dimerizing a last ethylene stream of said multiple ethylene streams to produce a dimerized olefin stream comprising ethylene dimers and oligomers; and
oligomerizing said dimerized olefin stream with an oligomerization catalyst to provide an oligomerized olefin stream.

14. (canceled)

15. The process of claim 13 further comprising cooling said first diluted ethylene stream immediately before dimerizing said first diluted ethylene stream.

16. The process of claim 14 further comprising diluting a second ethylene stream of said multiple ethylene streams with said first dimerized olefin stream to provide a second diluted ethylene stream and dimerizing said second diluted ethylene stream to produce a second dimerized olefin stream.

17. The process of claim 16 further comprising cooling said second diluted ethylene stream before dimerizing said second diluted ethylene stream.

18. The process of claim 14 further comprising taking a heavy olefin stream from said oligomerized olefin stream and saturating said heavy olefin stream to produce a saturated heavy stream and taking said paraffin stream from said saturated heavy stream.

19. A process for oligomerizing an olefin stream to produce distillate fuels comprising:
splitting an ethylene stream into multiple ethylene streams;
diluting a first ethylene stream of said multiple ethylene streams with a paraffin stream to provide a first diluted ethylene stream having no more than 25 wt % ethylene;
dimerizing a first diluted ethylene stream of said multiple olefin streams to produce a first dimerized olefin stream comprising ethylene dimers and oligomers; and
dimerizing a last olefin stream of said multiple olefin streams to produce a dimerized olefin stream comprising ethylene dimers and oligomers.

20. The process of claim 19 further comprising oligomerizing said dimerized olefin stream with an oligomerization catalyst to provide an oligomerized olefin stream.
